(19) United States
(12) Patent Application Publication
(10) Pub. No.: US 2019/0350888 A1
(43) Pub. Date: Nov. 21, 2019

(54) COMPOSITION FOR THE TREATMENT OF VEISALGIA

(71) Applicant: ph. AG, Zürich (CH)

(72) Inventor: Pedro SCHMIDT, Zürich (CH)

(21) Appl. No.: 16/322,764

(22) PCT Filed: Aug. 4, 2017
(86) PCT No.: PCT/EP2017/069805

§ 371 Date: Feb. 1, 2019

(30) Foreign Application Priority Data
Aug. 4, 2016 (EP) 16182785.2

(51) Int. Cl. A61K 31/198; A61K 31/205; A61K 31/355; A61K 31/616; A61K 31/7004; A61K 31/375; A61K 31/405; A61K 31/465; A61K 31/51; A61K 31/522; A61K 31/525; A61K 33/04; A61K 33/10; A61K 33/14; A61K 33/30; A61K 36/28; A61K 36/9068; A61K 38/06; A61K 45/06; A61K 9/00; A61P 25/32

(52) U.S. Cl. CPC A61K 31/198; A61P 25/32; A61K 31/355; A61K 31/616; A61K 31/7004; A61K 31/375; A61K 31/405; A61K 31/465; A61K 31/51; A61K 31/522; A61K 31/525; A61K 33/04; A61K 33/10; A61K 33/14; A61K 33/30; A61K 36/28; A61K 36/9068; A61K 38/06; A61K 45/06; A61K 9/0056; A61K 9/0095; A61K 31/205

(57) ABSTRACT

A composition for use in the therapy of veisalgia. The composition comprises at least one sugar compound, mineral salts, at least one compound for cell protection, at least one compound for promoting cell function, at least one compound for promoting detoxification, at least one neurotransmitter, at least one trace element, folic acid, optionally further trace elements and optionally a stimulating alkaloid. The composition contains no analgesic. A dosage of the composition, a therapy kit and an analgesic are also disclosed.

The invention concerns a composition for use in the therapy of veisalgia, a therapy kit for use in the treatment of veisalgia, and an analgesic for the treatment of veisalgia according to the preambles of the independent claims.

Veisalgia is the medical name for alcohol intoxication. It is often accompanied by a variety of symptoms such as headaches, nausea, dizziness, insomnia, dehydration and fatigue. The causes of these symptoms are very complex and not all details of the processes taking place in the body have been clarified. Nevertheless, some essential processes leading to the symptoms of veisalgia could be identified. Dehydration and demineralisation during heavy alcohol consumption, for example, contribute to the symptoms. This factor is further aggravated by the fact that those affected often fail to counteract the lack of fluids by consuming non-alcoholic beverages. Other causes of these symptoms include excessive stomach acidity, hypoglycaemia, acetaldehyde and acetate accumulation due to alcohol metabolism, oxidative stress, or degradation of NAD+ to NADH, which slows down the breakdown of alcohol in the body. However, which symptoms actually occur in the end, and also the severity of the symptoms, depends on many different factors, including the amount of alcohol or the height, weight and sex of the consuming person.

Therapeutic compositions are known which are intended to alleviate the symptoms at least partially. U.S. Pat. No. 3,829,569, for example, describes a composition with six essential ingredients: an analgesic, an antidepressant, a mild stomach remedy, a mild anesthetic, a metabolism enhancer and an antiacid. The drug is administered in the form of two capsules to be taken simultaneously, both capsules containing an analgesic. The partition into two capsules is made due to the high dosage of the individual components. The dosage stated in U.S. Pat. No. 3,829,569 is intended to significantly alleviate the symptoms of veisalgia. This pharmaceutical form has been shown to pose significant health risks.

The deficiency symptoms, which contribute to the formation of veisalgia, cannot be counteracted in this way.

A further composition for the treatment of the symptoms mentioned above is also described in WO 87/01285, wherein an analgesic is also co-administered here in each case.

It is therefore an object of the invention to overcome the disadvantages of the state of the art. In particular, one object of the invention is to provide a composition for use in the therapy of veisalgia that largely prevents or significantly reduces the occurrence of symptoms of veisalgia. The composition should be safe to use. It is a further object of the invention to provide a therapy kit for the treatment of veisalgia, which prevents the occurrence of symptoms as far as possible, but also allows symptomatic relief. It is also the object of the invention to provide an analgesic for the treatment of veisalgia for simultaneous or delayed administration with a further composition.

This problem is solved by the compositions, therapy kits and analgesics defined in the independent patent claims.

The invention concerns a composition for use in the therapy of veisalgia.

The composition comprises at least one sugar or sugar compound. The sugar or sugar compound is preferably selected from the group consisting of fructose, sucrose and glucose. A preferred glucose is D-glucose, also known as dextrose. Compounds from various sugars, such as fructose dextrose, are also conceivable. The sugar compounds prevent hypoglycaemia. Hypoglycaemia can lead to a series of negative processes that promote the development of veisalgia symptoms, such as fatigue, weakness and headaches. These symptoms are counteracted as far as possible by sugar intake.

The composition includes mineral salts. Preferred mineral salts are in particular a potassium salt, a sodium salt, a magnesium salt and a calcium salt. The use of potassium chloride, sodium citrate, magnesium carbonate and/or calcium carbonate is particularly preferred. This has the advantage that important minerals, which are washed out of the body due to alcohol consumption, are brought back into the body. These minerals are particularly important in relation to the nervous system and muscle system, and therefore also counteract the occurrence of numerous symptoms of veisalgia. However, different salts of a mineral can also be combined in the composition, for example magnesium carbonate, magnesium hydroxide and magnesium oxide. This strengthens, for example, the acid-binding properties and bioavailability of a mineral.

The composition comprises at least one compound for cell protection. Such a compound may in particular be an antioxidant. It has been shown to be beneficial to select at least one antioxidant from the following group: ascorbic acid, tocopherol, a glutathione precursor, glutathione and/or phosphatidylserine. Alternatively, it is also possible to use a combination of different antioxidants. For example, N-acyl-L-cysteine, L-cysteine and/or glutamic acid can be considered as glutathione precursors. Antioxidants are characterized by their properties as radical scavengers, reducing agents and by their pH-regulating properties. A combination of ascorbic acid (vitamin C), tocopherol (vitamin E) and N-acyl-L-cysteine (NAC) has proven to be particularly preferred. In particular, this minimizes oxidative stress and improves the feeling of well-being. Neurological cell protection, for example, is promoted by the addition of phosphatidylserine.

The composition further comprises at least one compound for supporting cell function. The compound is preferably selected from the group: nicotinamide (vitamin B3), nicotinic acid, riboflavin (vitamin B₂), pantothenic acid (vitamin B₅), pyridoxine (vitamin B₆), thiamine (vitamin B₁), vitamin B12. A combination of two or more of these compounds is particularly preferred. These compounds support the degradation of alcohol in the cells. Thiamine and vitamin B₁₂ contribute to neurocellular protection.

Furthermore, the composition includes at least one compound for promoting detoxification. This compound is in particular selected from the group: Betaine, Silybum marianum (milk thistle), Zingiberis rhizoma (ginger root), activated carbon, L-cysteine. However, it is also possible to use a combination of compounds promoting detoxification. These compounds are characterised by the fact that they can in particular counteract liver poisoning or promote the degradation of toxins in the liver.

The composition comprises at least one neurotransmitter. The neurotransmitter is preferably selected from the group: L-Tyrosine, L-Tryptophan, L-Phenylalanine, L-D-Phenylalanine (racemate), L-Glutamine, Dihydromyricetine, Choline bitartrate, Citrate. The term “neurotransmitter” also includes the precursors of neurotransmitters, i.e. substances that can be converted in the human body into the corresponding neurotransmitter. For example, L-tryptophan can be converted into serotonin and L-tyrosine into dopamine. These compounds have a mood-enhancing effect.

The composition comprises at least one trace element, in particular selenium and/or zinc; and folic acid. The use of all three trace elements has proven to be particularly advantageous. Other trace elements, such as biotin, manganese and/or molybdenum, can optionally be added to the composition. Trace elements are essential for maintaining a multitude of processes in the human body.

It may be desirable to add a stimulating alkaloid, in particular caffeine, to the composition. This particularly prevents symptoms of fatigue.

The composition does not contain any analgesic.

Such a composition for use in the therapy of veisalgia has the advantage that it reintroduces into the human body the nutrients that are lost through alcohol consumption or as a result thereof are required in increased quantities, thus reducing the symptoms of intoxication. Nutrients are generally understood to be compounds for maintaining biological processes in the human body that are relevant to health, in particular the compounds mentioned here. This composition also has the advantage that it can be taken preventively already before or during alcohol consumption, as it does not contain any analgesic and therefore there are no side effects with alcohol.

The composition may contain at least one further additive selected from the group: flavouring, colouring agents, decomposition accelerator, acidifier. A decomposition accelerator is a substance that improves the subsequent decomposition of the tablet. Sodium hydrogen carbonate and/or sodium carbonate are preferably used. However, corn and potato starch or polyvinylpyrrolidone are also conceivable. Citric acid is the preferred acidifier. More than one additive can also be used.

Such additives can on the one hand simplify the handling of the tablet and on the other hand improve the appearance and taste sensation.

The composition is preferably available as tablet, capsule, suspension, soluble powder, finished drink or depot formulation. The use of the composition in the form of effervescent tablets or soluble powder has proved to be particularly advantageous. The effervescent tablet or powder is preferably dissolved in 300 to 400 mL water for ingestion.

This composition is particularly easy and quick to take. Dissolving in water also has the advantage that a lack of liquid is compensated.

The invention also concerns a composition for use in the therapy of veisalgia. The composition comprises
at least one sugar or a sugar compound, in particular selected from the group: fructose, sucrose and glucose;
Mineral salts; in particular a potassium salt, a sodium salt, a magnesium salt and a calcium salt; and particularly preferably potassium chloride, sodium citrate, magnesium carbonate and/or calcium carbonate;
at least one compound for cell protection, in particular an antioxidant selected from the group: ascorbic acid, tocopherol, a glutathione precursor, in particular N-acyl-L-cysteine and/or L-cysteine; glutathione and/or phosphatidylserine;
at least one compound for supporting cell function, in particular selected from the group: nicotinamide, nicotinic acid, riboflavin, pantothenic acid, pyridoxine, thiamine, vitamin B12;
at least one compound for promoting detoxification, in particular betaine, Silybum marianum, Zingiberis rhizoma, activated carbon, L-cysteine;
at least one neurotransmitter, in particular selected from the group: L-tyrosine, L-tryptophan, L-phenylalanine, L-D-phenylalanine (racemate), L-glutamine, dihydromyricetine; choline bitartrate, citrate;
at least one trace element, in particular selenium and/or zinc;
Folic acid,
and optionally: other trace elements, in particular biotin, manganese and/or molybdenum;
optional: a stimulating alkaloid, especially caffeine; and is administered at least twice after alcohol consumption.

By at least twice administration, it is ensured that the lacking nutrients are supplied to the body again in sufficient quantities. A health-promoting nutrient concentration is maintained by taking the product at least twice. The symptoms associated with veisalgia are alleviated.

It has been shown to be particularly preferred if the composition is administered at least once directly after alcohol consumption and two more times within 12 hours. There should be a break of at least 2 hours between the administrations. The period of time from the first intake to the third intake should therefore be at least six hours and at most 12 hours.

This dosage has the advantage that the therapeutic effect is maintained even if there is a premature reduction in the nutrient concentration, due to pharmacokinetic properties and biological half-life.

The composition may be added at least once before or during alcohol consumption. Alternatively, it is also possible to administer the composition at least once before alcohol consumption and at least once during alcohol consumption.

The composition can therefore already be taken preventively or concomitantly. Taking it before alcohol consumption ensures that the body's nutrient reserves are replenished. The intake during alcohol consumption ensures that the nutrient concentration does not drop to a level that is hazardous to health. The symptoms caused by veisalgia are less noticeable. The absence of an analgesic means that there is no side effect due to the interaction of an analgesic with alcohol.

The composition may be administered together with an analgesic at least 4 hours after alcohol consumption. After 4 hours, the alcohol concentration in the blood is significantly reduced, so that taking an analgesic is less problematic.

The simultaneous intake of the composition and the analgesic has the advantage that the therapeutic effect is increased.

The composition may comprise
5 to 30 g of sugar or sugar compounds, preferably 12 to 20 g and especially preferred 16 to 18 g;
100 to 1500 mg of mineral salts, preferably 400 to 1300 mg and especially preferred 800 to 1100 mg;
10 to 900 mg of compounds for cell protection, preferably 50 to 300 mg and especially preferred 90 to 200 mg;
0.5 to 7 g of the neurotransmitter, preferably 3 to 7 g of the neurotransmitter and especially preferred 3 to 5 g;
1 to 30 mg of compounds supporting cell function, preferably 15 to 30 mg and especially preferred 25 to 30 mg;
200 to 2500 mg of compounds promoting detoxification, preferably 200 to 1200 mg and particularly preferred 500 to 800 mg;
1 to 7 mg of trace element and folic acid, preferably 5 to 6 mg; and
optionally: 2 to 10 mg biotin; 1 to 6 mg manganese; 50 to 300 mg molybdenum; and/or 20 to 100 mg caffeine.

A combination of the individual compounds in the broadest quantity range is a preferred embodiments of the invention.

This information refers to the one-time intake of the composition. In total, up to 50 g of sugar or sugar compounds, 6 g of mineral salts, 6 g of compounds for cell protection, 14 g of neurotransmitters, 500 mg of compounds for supporting cell function, 6 g of compounds for promoting detoxification, 1 mg folic acid and 50 mg trace elements can be used per therapy. The individual dosages and/or compositions of the individual compounds to be administered before, during and after alcohol consumption may be the same or vary. The individual compounds are preferably dosed in such a way that they are in the range of the recommended daily dose, based on the single dose or total dose. For example, the total dose of magnesium should not exceed 400 mg. The overdosing of water-binding substances can have a laxative effect. The dosage of vitamin C in a single dose is preferably in the range of the recommended daily dose.

Furthermore, the invention concerns a therapy kit for use in the treatment of veisalgia. The therapy kit comprises at least two components for separate administration. A first component is a composition of
at least one sugar or a sugar compound, in particular selected from the group: fructose, sucrose and glucose;
Mineral salts; in particular a potassium salt, a sodium salt, a magnesium salt and a calcium salt; and particularly preferred potassium chloride, sodium citrate, magnesium carbonate and/or calcium carbonate;
at least one compound for cell protection, in particular an antioxidant selected from the group: ascorbic acid, tocopherol; a glutathione precursor, in particular N-acyl-L-cysteine and/or L-cysteine; glutathione and/or phosphatidylserine;
at least one compound for supporting cell function, in particular selected from the group: nicotinamide, nicotinic acid, riboflavin, pantothenic acid, pyridoxine, thiamine, vitamin B12;
at least one compound for promoting detoxification, in particular betaine, Silybum marianum, Zingiberis rhizoma, activated carbon, L-cysteine;
at least one neurotransmitter, in particular selected from the group: L-tyrosine, L-tryptophan, L-phenylalanine, L-D-phenylalanine (racemate), L-glutamine, dihydromyricetine; choline bitartrate, citrate;
at least one trace element, in particular selenium and/or zinc;
folic acid, and
optionally further trace elements, in particular biotin, manganese and/or molybdenum; and
optionally: a stimulating alkaloid, especially caffeine and does not contain any analgesic.

The second component is an analgesic and may alternatively contain an antacid and/or a stimulating alkaloid, preferably caffeine.

The composition of the first components may have the quantities of the compounds described above. The kit may include several individual doses of the first component and the second component, differing in the composition of the compounds and/or the dosages of the individual compounds.

The second component may comprise 100 to 1000 mg of the analgesic in a dose to be administered once. Optionally, the second component may also contain 100 to 1000 mg of an antacid and/or 20 to 100 mg of a stimulating alkaloid. There may also be different doses of an analgesic in the kit.

The kit is characterized by the fact that the composition and the analgesic for the treatment of veisalgia are available simultaneously and in sufficient quantities. But also a separate intake of the components is possible. A component can also be omitted if it is not needed. With the therapy kit, the treatment can be optimally adapted to the severity of the veisalgia as well as the symptoms. It was also surprisingly found that side effects of the analgesic can be minimized if the first component is taken almost simultaneously with the analgesic. The first component thus has an additional positive effect on the second component.

The analgesic is preferably selected from the group: acetylsalicylic acid, ibuprofen, acetaminophen, fenoprofen, mefenamic acid, naproxen, codeine or tolfenamic acid.

It has proved beneficial to use well investigated and standardised analgesics. Analgesics with caffeine are known and easily accessible. Their interactions and side effects are well documented and can therefore be taken into account for the present invention as part of routine measures. Commercial products suitable according to the present invention are, for example, Contraschmerz® plus or Alcacyl® Extra.

The antacid may be selected from the group consisting of aluminium hydroxide, magnesium hydroxide, calcium carbonate, magnesium carbonate or aluminium-magnesium silicate hydrate. A combination of different antacids is also possible.

The use of an antacid reduces the risk of excessive stomach acidity. This may be particularly important when taking ibuprofen. Ibuprofen is known for its stomach irritating effect. At the same time, the antacid can also increase the bioavailability of minerals such as calcium or magnesium.

The first and second components of the therapy kit can be administered together or sequentially at least 4 hours after alcohol consumption. For example, the first component can be administered 4 hours after alcohol consumption and the second component an hour later. A delayed intake of up to 2 hours is conceivable.

The advantage of simultaneous administration is that the therapeutic effect is improved. The advantage of sequential administration is that administration can be effected depending on the symptoms to be treated.

Further joint or delayed administration of the components may take place at least 6 hours after alcohol consumption. This also results in an improved therapeutic effect.

Alternatively, the first component may be administered at least once before and during alcohol consumption. It is also possible that the composition is additionally administered only before alcohol consumption or only during alcohol consumption.

Taking the first component before alcohol consumption has the advantage that the nutrient reserves in the body can be replenished. Administration during alcohol consumption reduces the loss of these nutrients during this time. Depending on the amount of consumed alcohol, this can prevent the occurrence of veisalgia or reduce the severity of the symptoms.

The invention also concerns an analgesic for the treatment of veisalgia by simultaneous or sequential administration with a composition as described above.

The analgesic is preferably acetylsalicylic acid, ibuprofen, acetaminophen, fenoprofen, mefenamic acid, naproxen, codeine or tolfenamic acid.

A therapeutic effect of the analgesic can be improved by a joint administration with the composition. The sequential administration enables the treatment to be adapted to the course of the disease.

### EXAMPLE 1

Example 1 shows a preferred dosage for a first component and a second component for administration after alcohol consumption.

| | [mg] |
|---|---|
| Composition | |
| (first component) | |
| effervescent tablet or soluble | |
| powder in 330 mL water | |
| sugar compound | |
| fructose | 10000 |
| dextrose* | 5000 |
| minerals | |
| potassium chloride | 660 |
| sodium citrate | 200 |
| magnesium carbonate | 125 |
| calcium carbonate | 266 |
| detoxification | |
| betaine hydrochloride | 500 |
| Silybum marianum | 133 |
| Zingiberis rhizoma | 500 |
| cell protection | |
| vitamin C | 80 |
| L-cysteine | 300 |
| N-acetyl-L-cysteine | 200 |
| glutathione | 200 |
| vitamin E | 12 |
| phosphatidylserine | 100 |
| neurotransmitters | |
| L-tryptophan | 500 |
| L-tyrosine | 166 |
| L-Glutamine | 3000 |
| L-D-phenylalanine | 333 |
| (racemate) | |
| dihydromyricetine | 300 |
| cell function | |
| Vitamin B₂ (Riboflavin) | 1.4 |
| Vitamin B₁ (thiamine) | 1.1 |
| Vitamin B₃ (Nicotinamide)** | 18 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| folic acid | 0.2 |
| analgesic | |
| (second component) | |
| Orally ingestible tablet | |
| acetylsalicylic acid | 500 |
| caffeine | 50 |

*Alternatively, 0 g dextrose may be present.
**Alternatively, 16 mg or 17 mg are also possible

The first component in example 1 can be taken alone, together with the second component or staggered with the second component. Alternatively, the first component may also be administered alone before and/or during alcohol consumption.

### EXAMPLE 2

Example 2 shows another preferred dosage for a first component and a second component. The first component may be taken before or immediately after alcohol consumption, preferably without the second component. The second intake can be taken approximately 4 hours after drinking alcohol or after getting up (approximately 8 hours after drinking alcohol), preferably with the second component. A third dose can be taken either 3 hours after the second dose or after getting up (approx. 8 hours later). Preferably the third intake is also made with the second component. Components 1 and 2 may be available as therapy kit.

| | [mg] |
|---|---|
| Composition | |
| (first component) | |
| effervescent tablet or soluble | |
| powder in 330 mL water | |
| sugar compound | |
| fructose | 12000 |
| glucose | 6000 |
| minerals | |
| potassium chloride | 660 |
| magnesium carbonate | 125 |
| calcium carbonate | 266 |
| detoxification | |
| L-cysteine | 300 |
| Trimethylglycine (Betaine) | 500 |
| cell protection | |
| vitamin C | 80 |
| vitamin E | 12 |
| phosphatidylserine | 100 |
| neurotransmitters | |
| L-tryptophan | 80 |
| L-tyrosine | 500 |
| L-Glutamine | 2500 |
| Choline Bitartrate or Citrate | 100 |
| cell function | |
| Vitamin B₂ (Riboflavin) | 1.4 |
| Vitamin B₁ (thiamine) | 1.1 |
| Vitamin B₃ (Nicotinamide) | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| folic acid | 0.2 |
| additives | |
| sodium bicarbonate | 600 |
| citric acid | 1500 |
| analgesic | |
| (second component) | |
| Orally ingestible tablet | |
| acetylsalicylic acid | 500 |
| caffeine | 60 |

### EXAMPLE 3

Example 3 shows three different dosages of the individual compositions to be administered sequentially when consuming alcohol. The first dose is preferably taken before or immediately after alcohol consumption without the second component. The second dosage can be taken approximately 4 hours after alcohol consumption or after getting up (approximately 8 hours after alcohol consumption), preferably with a first composition of a second component. The third dose can be taken either 3 hours after the second intake or after getting up (approx. 8 hours later), preferably with a second composition for the second component. The individual doses may be available as a therapy kit comprising the three differently dosed compositions and the differently dosed analgesic. The composition of the individual dosages is preferably as follows:

| | [mg] |
|---|---|
| Composition of the first dosage | |
| (1) | |
| (first component) | |
| effervescent tablet or soluble | |
| powder in 330 mL water | |
| sugar compound | |
| fructose | 16000 |
| glucose | 2000 |
| minerals | |
| potassium chloride | 660 |
| magnesium carbonate | 125 |
| calcium carbonate | 266 |
| detoxification | |
| L-cysteine | 500 |
| Sylybum marianum | 250 |
| cell protection | |
| vitamin C | 80 |
| vitamin E | 12 |
| phosphatidylserine | 200 |
| neurotransmitters | |
| L-tryptophan | 200 |
| L-tyrosine | 500 |
| L-Glutamine | 4000 |
| dihydromyricetine | 200 |
| cell function | |
| Vitamin B₂ (Riboflavin) | 1.4 |
| Vitamin B₁ (thiamine) | 1.1 |
| Vitamin B₃ (Nicotinamide) | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| folic acid | 0.2 |
| additives | |
| sodium bicarbonate | 600 |
| citric acid | 1500 |
| Composition of the second | |
| dosage (2) | |
| (first component) | |
| effervescent tablet or soluble | |
| powder in 330 mL water | |
| sugar compound | |
| fructose | 2000 |
| glucose | 16000 |
| minerals | |
| potassium chloride | 660 |
| magnesium carbonate | 125 |
| calcium carbonate | 266 |
| detoxification | |
| L-cysteine | 300 |
| Sylybum marianum | 250 |
| cell protection | |
| vitamin C | 80 |
| vitamin E | 12 |
| phosphatidylserine | 50 |
| neurotransmitters | |
| L-tryptophan | 20 |
| L-tyrosine | 500 |
| L-Glutamine | 2000 |
| dihydromyricetine | 100 |
| cell function | |
| Vitamin B₂ (Riboflavin) | 1.4 |
| Vitamin B₁ (thiamine) | 1.1 |
| Vitamin B₃ (Nicotinamide) | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| folic acid | 0.2 |
| additives | |
| sodium bicarbonate | 600 |
| citric acid | 1500 |
| Composition of the third dose | |
| (3) | |
| (first component) | |
| effervescent tablet or soluble | |
| powder in 330 mL water | |
| sugar compound | |
| fructose | 9000 |
| glucose | 9000 |
| minerals | |
| potassium chloride | 660 |
| magnesium carbonate | 125 |
| calcium carbonate | 266 |
| detoxification | |
| L-cysteine | 100 |
| Sylybum marianum | 250 |
| cell protection | |
| vitamin C | 80 |
| vitamin E | 12 |
| phosphatidylserine | 50 |
| neurotransmitters | |
| L-tryptophan | 20 |
| L-tyrosine | 500 |
| L-Glutamine | 1000 |
| dihydromyricetins | 100 |
| cell function | |
| Vitamin B₂ (Riboflavin) | 1.4 |
| Vitamin B₁ (thiamine) | 1.1 |
| Vitamin B₃ (Nicotinamide) | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| folic acid | 0.2 |
| additives | |
| sodium bicarbonate | 600 |
| citric acid | 1500 |
| An analgesic-first composition | |
| for administration with (2) | |
| (second component) | |
| Orally ingestible tablet | |
| acetylsalicylic acid | 500 |
| caffeine | 50 |
| An analgesic second composition | |
| for administration with | |
| (3) | |
| (second component) | |
| Orally ingestible tablet | |
| acetylsalicylic acid | 1000 |
| caffeine | 100 |

### EXAMPLE 4

Example 4 shows different dosages and composition for the sequential administration in the case of consumption of alcohol. The first composition and dosage is preferably taken before or after alcohol consumption without the second component. The second composition and dosage can be taken approximately 4 hours after alcohol consumption or after getting up (approximately 8 hours after alcohol consumption), preferably with a first composition of a second component. The third composition and dosage may be taken optionally 3 hours after the second ingestion or after getting up (about 8 hours later), preferably with a second composition for the second component. The individual compositions and dosages may be available as a therapy kit comprising the three differently dosed compositions and the differently composed analgesics. The composition of the individual dosages is preferably as follows:

| | [mg] |
|---|---|
| Composition of the first dosage | |
| (1) | |
| (first component) | |
| effervescent tablet or soluble | |
| powder in 330 mL water | |
| sugar compound | |
| fructose | 16000 |
| minerals | |
| potassium chloride | 660 |
| magnesium carbonate | 125 |
| calcium carbonate | 266 |
| detoxification | |
| L-cysteine | 500 |
| Sylybum marianum | 250 |
| Trimethylglycine (Betaine) | 1500 |
| cell protection | |
| vitamin C | 80 |
| vitamin E | 12 |
| phosphatidylserine | 200 |
| neurotransmitters | |
| L-tryptophan | 200 |
| L-tyrosine | 500 |
| L-Glutamine | 1000 |
| dihydromyricetins | 200 |
| cell function | |
| Vitamin B₂ (Riboflavin) | 1.4 |
| Vitamin B₁ (thiamine) | 1.1 |
| Vitamin B₃ (Nicotinamide)** | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| folic acid | 0.2 |
| additives | |
| sodium bicarbonate | 600 |
| citric acid | 1500 |
| Composition of the second | |
| dosage (2) | |
| (first component) | |
| effervescent tablet or soluble | |
| powder in 330 mL water | |
| sugar compound | |
| fructose | 12000 |
| glucose | 4000 |
| minerals | |
| potassium chloride | 660 |
| magnesium carbonate | 125 |
| calcium carbonate | 266 |
| detoxification | |
| L-cysteine | 400 |
| Sylybum marianum | 250 |
| Zingiberis rhizoma | 300 |
| activated carbon | 600 |
| cell protection | |
| vitamin C | 80 |
| vitamin E | 12 |
| phosphatidylserine | 200 |
| neurotransmitters | |
| L-tryptophan | 100 |
| L-tyrosine | 500 |
| L-Glutamine | 5000 |
| dihydromyricetins | 400 |
| Choline Bitartrate or | 100 |
| Citrate | |
| cell function | |
| Vitamin B₂ (Riboflavin) | 1.4 |
| Vitamin B₁ (thiamine) | 1.1 |
| Vitamin B₃ (Nicotinamide)** | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 0.018 |
| zinc | 5 |
| additives | |
| sodium bicarbonate | 600 |
| citric acid | 1500 |
| Composition of the third dose | |
| (3) | |
| (first component) | |
| effervescent tablet or soluble | |
| powder in 330 mL water | |
| sugar compound | |
| glucose | 16000 |
| minerals | |
| potassium chloride | 660 |
| magnesium carbonate | 125 |
| calcium carbonate | 266 |
| detoxification | |
| Zingiberis rhizoma | 500 |
| Sylybum marianum | 250 |
| activated carbon | 600 |
| cell protection | |
| vitamin C | 80 |
| vitamin E | 12 |
| neurotransmitters | |
| L-tyrosine | 500 |
| Choline Bitartrate or Citrate | 200 |
| dihydromyricetine | 200 |
| cell function | |
| Vitamin B₂ (Riboflavin) | 1.4 |
| Vitamin B₁ (thiamine) | 1.1 |
| Vitamin B₃ (Nicotinamide) | 16 |
| Vitamin B₁₂ (cobolamine) | 0.0025 |
| Vitamin B₆ (pyridoxine) | 1.4 |
| Vitamin B₅ (pantothenic acid) | 6 |
| trace elements | |
| selenium | 5 |
| zinc | 0.2 |
| additives | |
| sodium bicarbonate | 800 |
| An analgesic-first composition | |
| for administration with (2) | |
| (second component) | |
| Orally ingestible tablet | |
| acetylsalicylic acid | 500 |
| aluminium hydroxide | 600 |
| An analgesic second composition | |
| for administration with | |
| (3) | |
| (second component) | |
| Orally ingestible tablet | |
| acetylsalicylic acid | 1000 |
| caffeine | 100 |
| aluminium hydroxide | 600 |

The invention is explained in more detail below on the basis of the figure, which is merely a dosage example. It shows

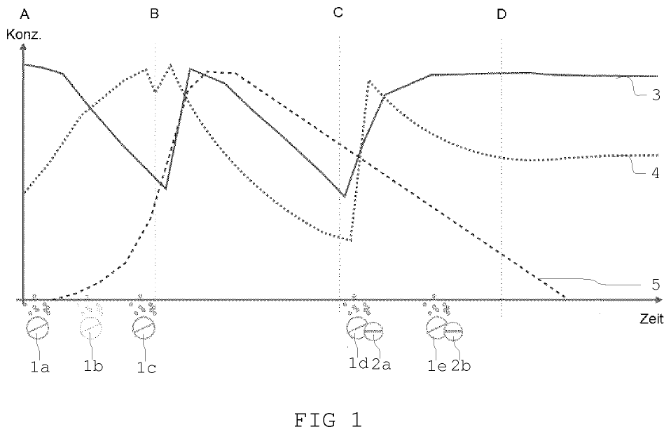

FIG. 1: A dosage of the composition according to the invention and a schematic representation of the variation of concentration of the substances present in the body.

FIG. 1 schematically shows the concentration curve of the substances present in the body depending on a dosage of the composition according to the invention. Curve 3 indicates the variation of minerals during drinking, characterized by the time interval A-B, during sleep, characterized by the time interval B-C, during the occurrence of veisalgia symptoms, characterized by the time interval C-D, and after veisalgia D. Curve 4 accordingly indicates the variation of blood sugar, and curve 5 indicates the relative concentration of toxins in the blood. Toxic substances are in particular degradation products due to alcohol metabolism, such as acetaldehyde and acetate. A dosage form of the composition 1a is taken before alcohol consumption A. The concentration of minerals 3 in the blood reaches a maximum value. During alcohol consumption A-B, the concentration of mineral substances 3 decreases. The blood sugar level 4 rises, also due to the intake of alcoholic beverages. The concentration of toxins 5 continues to rise. Optionally, during alcohol consumption A-B, the composition 1b can be taken once more. After alcohol consumption B, the composition 1c is taken an additional time. As a result, the concentration of mineral substances 3 increases again in the initial phase of sleep phase B-C, but drops again later due to consumption in biological processes, particularly in connection with the degradation of toxins 5. The concentration of blood sugar 4 also drops again after a short increase. Due to the alcohol metabolism, the concentration of toxins 5 in the blood increases to a maximum value during the sleep phase B-C. The concentration of toxins 5 is then steadily reduced. The relationship between the degradation of toxins 5 and the loss of nutrients 3 is illustrated by the almost parallel slope of the curve. Immediately after waking up C, the composition 1d is taken together with an analgesic 2a in order to accelerate the treatment of veisalgia, to counteract the development of symptoms and to achieve the quickest possible relief. By taking the composition 1d the blood sugar 4 rises again, also the concentration of mineral substances 3 increases. 2 hours after waking up C the composition 1e is taken once more with an analgesic 2b. This also supports the degradation of toxins 5. After the healing of veisalgia D, the concentration of mineral substances 3 and blood sugar 4 reaches an almost constant value. The toxins 5 are almost completely degraded.

1-15. (canceled)

16. A composition for use in the therapy of veisalgia comprising:
at least one sugar or a sugar compound;
mineral salts;
at least one compound for cell protection, in particular an antioxidant selected from the group: ascorbic acid; tocopherol; glutathione precursor, in particular N-acyl-L-cysteine, L-cysteine and/or glutamic acid; glutathione and/or phosphatidyl serine;
at least one compound for supporting cell function selected from the group: nicotinamide; nicotinic acid; riboflavin; pantothenic acid; pyridoxine; thiamine; vitamin B12;
at least one compound for promoting detoxification selected from the group: betaines; Silybum marianum; Zingiberis rhizome; activated carbon; L-cysteine;
at least one neurotransmitter;
at least one trace element, in particular selenium and/or zinc;
folic acid;
optionally further trace elements, in particular biotin, manganese and/or molybdenum; and
optionally a stimulating alkaloid, in particular caffeine;
wherein the composition does not contain an analgesic.

17. The composition according to claim 16, wherein the composition contains at least one further additive selected from the group consisting of aroma; colorants; decomposition accelerator; and acidifier.

18. The composition according to claim 16, wherein the composition is in the form of a tablet;
a capsule;
a suspension;
a soluble powder;
a ready-to-drink beverage, or
a depot formulation.

19. The composition according to claim 17, wherein the composition is in the form of a tablet;
a capsule;
a suspension;
a soluble powder;
a ready-to-drink beverage, or
a depot formulation.

20. A composition for use in the therapy of veisalgia, the composition comprising:
at least one sugar or a sugar compound;
mineral salts;
at least one compound for cell protection;
at least one compound for supporting cell function;
at least one compound for promoting detoxification;
at least one neurotransmitter;
at least one trace element;
folic acid;
optionally further trace elements;
optionally a stimulating alkaloid for administration at least twice after alcohol consumption.

21. The composition according to claim 20, wherein the administration is at least once directly after alcohol consumption and two further times within 12 hours, with a break of at least 2 hours between the intakes.

22. The composition according to claim 20, for at least one additional administration of the composition before and/or during alcohol consumption.

23. The composition according to claim 21, for at least one additional administration of the composition before and/or during alcohol consumption.

24. The composition according to claim 20, for administration of the composition at least 4 hours after alcohol consumption together with an analgesic.

25. The composition according to claim 16, the composition comprising:
5 to 30 g of sugar or sugar compounds;
100 to 1500 mg of mineral salts;
10 to 900 mg of compounds for cell protection;
0.5 to 7 g of neurotransmitters;
1 to 30 mg of compounds supporting cell function;
200 to 2500 mg of compounds for promoting detoxification;
1 to 7 mg of trace elements and folic acid; and
optionally 2 to 10 mg biotin; 1 to 6 mg manganese; 0-300 mg molybdenum; and/or 20-100 mg caffeine.

26. The composition according to claim 20, the composition comprising:
5 to 30 g of sugar or sugar compounds;
100 to 1500 mg of mineral salts;
10 to 900 mg of compounds for cell protection;
0.5 to 7 g of neurotransmitters;
1 to 30 mg of compounds supporting cell function;
200 to 2500 mg of compounds for promoting detoxification;
1 to 7 mg of trace elements and folic acid; and
optionally 2 to 10 mg biotin; 1 to 6 mg manganese; 0-300 mg molybdenum; and/or 20-100 mg caffeine.

27. A therapy kit for use in the treatment of veisalgia, comprising at least two separately administrable components, wherein the first component is a composition comprising:
at least one sugar or a sugar compound;
mineral salts;
at least one compound for cell protection;
at least one compound for supporting the cell function;
at least one compound for promoting detoxification;
at least one neurotransmitter;
at least one trace element;
folic acid:
optionally further trace elements;
optionally a stimulating alkaloid;
and does not contain an analgesic;
and wherein the second component contains an analgesic and optionally an antacid and/or optionally a stimulating alkaloid, preferably caffeine.

28. The therapy kit according to claim 27, wherein the analgesic is selected from the group consisting of:
acetylsalicylic acid;
ibuprofen;
acetaminophen;
fenoprofen;
mefenamic acid;
naproxen;
codeine; and
tolfenamic acid.

29. The therapy kit according to claim 28, wherein the antacid is selected from the group consisting of:
aluminum hydroxide;
magnesium hydroxide;
calcium carbonate;
magnesium carbonate; and
aluminum magnesium silicate hydrate.

30. The therapy kit according to claim 28, for the concomitant or sequential administration of the components at least 4 hours after alcohol consumption.

31. The therapy kit according to claim 28, for concomitant or sequential administration of the components at least 6 hours after alcohol consumption.

32. The therapy kit according to claim 28, for the administration of the first component at least once before alcohol consumption and/or during alcohol consumption.

33. An analgesic for the treatment of veisalgia by concomitant or sequential administration with a composition according to claim 16.
